(19) United States
(12) Patent Application Publication
(10) Pub. No.: US 2019/0351089 A1
(43) Pub. Date: Nov. 21, 2019

(54) AEROSOL SYSTEM FOR ELIMINATING BATHROOM ODORS

(71) Applicant: Zenex International, Inc., Cleveland, OH (US)

(72) Inventors: Sean Biros, Thompson, OH (US); George Kneier, Brecksville, OH (US); Frank Hanzl, Peninsula, OH (US)

(21) Appl. No.: 16/101,606

(22) Filed: Aug. 13, 2018

Related U.S. Application Data
(60) Provisional application No. 62/674,668, filed on May 22, 2018.
(60) Provisional application No. 62/673,492, filed on May 18, 2018.

(51) Int. Cl. A61L 9/14; A61L 9/01; E03D 9/00

(52) U.S. Cl. CPC A61L 9/14; E03D 9/00; A61L 9/01

(57) ABSTRACT

A biologically-active aerosol spray composition for eliminating the persistent noxious odors commonly found in residential and commercial bathrooms comprises a mixture of endospores of one or more beneficial bacteria capable of digesting dried urine without producing noxious odors and a carrier liquid for the endospores, wherein the carrier liquid comprises acetone.

### RELATED APPLICATIONS

This application claims the benefit of and priority to U.S. Provisional Application Ser. No. 62/674,668, filed May 22, 2018, and U.S. Provisional Application No. 62/673,492 filed May 18, 2018, which are incorporated herein by reference in their entirety.

### BACKGROUND

Persistent unpleasant odor can be a recurrent problem in bathrooms, both residential and commercial. Normally, this is caused by small amounts of waste, primarily dried urine but also including food wastes, mold, mildew, feces and other filth, contaminating the floors and other hard surfaces surrounding toilets, urinals and the like. In addition, these wastes can be spread to surrounding areas, both conspicuous and non-conspicuous spaces, by foot traffic, mop, bucket, hand contamination, etc. Naturally-occurring bacteria in the air, when activated by water, then digest these wastes, generating decomposition products exhibiting noxious odors such as ammonia and thiols.

It has already been proposed to deal with this problem by periodically applying to these hard surfaces biologically-active compositions containing bacteria, enzymes or both which are capable of digesting these wastes without producing noxious odors (“beneficial bacteria”). Unfortunately, most of these products are difficult to use, as a practical matter. For example, those available in the form of powders as well as those available in the form of bulk liquids are normally applied by hand and hence are difficult to use.

Meanwhile, those which are applied by mechanical pump spray devices normally contain water, which can create a slippage hazard. Similarly, those which are applied in the form of aerosol sprays containing ethanol or other alcohol also can create a slippage hazard. In addition, the alcohol can also react with materials already present on the hard surfaces such as floor wax, polish or the like causing additional problems.

### SUMMARY OF THE INVENTION

In accordance with this invention, it has been found that these problems can be eliminated essentially completely by applying these beneficial bacteria by means of an aerosol spray system in which the biologically-active composition being applied comprises bacterial endospores and a carrier liquid for these endospores comprising acetone.

Thus, this invention provides a biologically-active composition comprising a mixture of endospores of one or more beneficial bacteria capable of digesting dried urine without producing noxious odors and a carrier liquid for the endospores, wherein the carrier liquid comprises acetone.

In addition, this invention also provides a new aerosol spray dispenser comprising a pressurized aerosol canister comprising a container, an aerosol valve, and a pressurized aerosol composition inside the container comprising the above biologically-active composition and an aerosol propellant.

In addition, this invention also provides a process for eliminating the persistent unpleasant odors often found in commercial and residential bathrooms, the process comprising automatically and periodically discharging into the atmosphere of the bathroom being treated the above biologically-active composition in such a way that the endospores in this composition deposit on hard surfaces in the bathroom carrying dried urine while the acetone and propellant in this composition do not.

### DETAILED DESCRIPTION

### Beneficial Bacterial Endospores

As indicated above, the inventive biologically-active composition contains endospores of beneficial bacteria, i.e., bacteria capable of digesting dried urine without producing noxious odors. As well-understood in the art, bacterial endospores are living creatures in a dormant state which become biologically active when exposed to appropriate activating conditions, which in the case of this invention normally include water as well as the proteins and other chemicals found in dried urine.

Spore-forming bacteria capable of digesting dried urine without producing noxious odors are well-known and available from a variety of different commercial sources, examples of which include Envera, LLC, of West Chester, Pa.

Typically, these spore-forming bacteria are members of the phylum Firmicutes (generally previously defined as Bacillus species) as listed in Bergey's Manual of Systematic Bacteriology, Second Edition (2009), hereby incorporated by reference in its entirety. Examples include B. alcalophilus, B. alvei, B. amyloliquefaciens, B. aneurinolyticus, B. aquaemaris, B. atrophaeus, B. boronophilus, B. brevis, B. caldolyyicus, B. centrosporus, B. cereus, B. circulans, B. clausii, B. coagulans, B. firmus, B. flavothermus, B. fusiformis, B. globigii, B. infernus, B larvae, B. laterosporus, B. lentimorbus, B. lentus, B. licheniformis, B. megaterium, B. mesentericus, B. mucilaginosus, B. mycoides, B. natto, B. pantothenicus, B. popilliae, B. polymyxa, B. pseudoanthracis, B. pumilus, B. schlegelii, B. simplex, B. sphaericus, B. sporothermodurans, B. stearothermophilus, B. subtilis, B. thermoglucosidasius, B. thuringiensis, B. vulgatis, B. weihenstephanensis, as well as genetically modified variants of such species. Preferred species include B. amyloliquefaciens, B. brevis, B. cereus, B. circulans, B. clausii, B. coagulans, B. firmus, B. laterosporus, B. lentus, B. licheniformis B. megaterium, B. polymyxa, B. pumilus, B. simplex, B. sphaericus, B. stearothermophilus, B. subtilis, and B. thuringiensis.

### Biologically-Active Compositions

In addition to endospores of beneficial bacteria, the inventive biologically-active composition also contains acetone or analog as a carrier liquid for the endospores. In this context, an analog of acetone will be understood to mean any other organic liquid which has a vapor pressure which is greater than the vapor pressure of acetone at room temperature and, in addition, is designated as a “VOC exempt” compound by the California Air Resources Board, a division of the California Environmental Protection Agency.

As indicated above, the bacterial endospores which are useful in this invention will become biologically active when contacted with water if an appropriate “food source” is also present, in this case the proteins and/or other ingredients found in dried urine. In addition, water represents a slippage hazard, since it evaporates slowly due to its relatively low vapor pressure. Ethanol also represents a slippage problem for the same reason. To avoid this slippage problem and, in addition, to improve the shelf life of the inventive biologically-active composition, this composition is preferably made with little or no water or ethanol.

In this context, “little or no water or ethanol” means that the total concentration of both water and ethanol in the inventive biologically-active composition is less than 20 wt. %, based on the weight of the composition as a whole. In this context, the “weight of the composition as a whole” will be understood to mean the combined weights of its biological endospores, its carrier liquid and any germinative compound and other additional ingredients that might be present, but excluding any liquid propellant that might be used when the composition is included in a pressurized aerosol spray system. Preferably, the combined concentration of water plus ethanol in the inventive biologically-active composition is less than 10 wt. %, less than 5 wt. % or less than even 2 wt. % on the same basis. Even more preferably, the inventive biologically-active composition contains no more than trace amounts of water and ethanol. Most preferably, the inventive biologically-active composition is entirely free of water and ethanol.

Dormant, water-free, bacterial endospores in fine powdery form can be conveniently produced by various different drying processes including spray-drying, freeze-drying, air drying, drum drying and the like. See, the above-noted U.S. Pat. No. 9,447,376. Although these water-free fine powdery products can be used directly, it is more convenient and effective when making and using sprayable aerosol compositions if these endospores are supplied in the form of a suspension of the endospores in an appropriate carrier liquid.

In accordance with this invention, this carrier liquid comprises acetone or analog. Because of its low vapor pressure, acetone evaporates before reaching the floor or other hard surface to which the inventive biologically-active composition is applied. Therefore, the slippage hazard that occurs when water or ethanol is used as the carrier liquid is entirely avoided. In addition, problems that can occur when ethanol reacts with, or otherwise affects, polishes, waxes or other floor treatments are also entirely avoided.

### Other Conventional Ingredients

In addition to the above ingredient, the inventive biologically-active composition can also contain any other ingredient which is conventionally included in similar compositions. Examples include perfumes, deodorant compounds, astringent salts, antioxidants, and the like.

### Ingredient Concentrations

The concentration of ingredients in the inventive biologically-active composition can vary widely, and essentially any concentration can be used which will enable the bacterial endospores of this invention to be deposited on hard bathroom surfaces in the manner described below. Normally, the concentration of bacterial endospores in the inventive biologically-active composition will be at least 1 wt. % but no more than 10 wt. %, based on the composition as a whole. Preferably, the concentration of bacterial endospores in the inventive biologically-active composition will be from 2 to 8 wt. %, more typically, from 3 to 7 wt. % or even 4 to 6 wt. % on the same basis.

Similarly, if the inventive biologically-active composition also contains additional ingredients such as perfumes, deodorants, astringents, antioxidants, the combined concentrations of all these additional ingredients will normally not exceed 10 wt. %. More typically, this concentration will not exceed 8 wt. %, 7 wt. % or even 6 wt. %, on the same basis.

### Aerosol Spray Dispenser

The inventive biologically-active composition is designed to be administered by means of an aerosol spray dispenser. As appreciated by those skilled in the art, an aerosol spray dispenser normally comprises a container, an aerosol valve, an active ingredient and a propellant capable of propelling the active ingredient out of the container in the form of a spray when the aerosol valve is actuated. These dispensers are well-known and any such system can be used. See, for example, U.S. Pat. Nos. 5,348,731, 6,126,930 and U.S. 2007/0015675, which describe these dispensers in detail. The disclosures of each of these references are also incorporated herein by reference in their entireties.

As well appreciated in the art, a wide range of different chemical compounds can be used as propellants in aerosol spray dispensers. Examples include compressed gas propellants such as compressed air, nitrogen, nitrous oxide and carbon dioxide, as well as liquefied gas propellants such as low molecular weight hydrocarbons, e.g., propane, isobutane, isobutene, n-butane, and dimethyl ether, and hydrofluorocarbons such as HFC 152a and HFC 134a. Mixtures of these propellants can also be used.

### Aerosol Systems

In accordance with a preferred embodiment of this invention, the inventive biologically-active composition is automatically and periodically dispensed into the atmosphere of the bathroom being treated. For this purpose, the above-described aerosol spray dispenser can be combined with an automatic dispensing assembly which is structured to automatically and repeatedly actuate the aerosol valve of the dispenser in accordance with a predetermined time pattern. For example, this assembly can be designed to automatically and repeatedly actuate this valve at a predetermined interval of time, e.g., once every 5 minutes, once every 10 minutes, once every 15 minutes, once every 20 minutes, once every 25 minutes, once every 30 minutes, once every 45 minutes, once every 1 hour, once every 1.5 hours, once every 2 hours, etc. and, in addition, to cause the biologically-active composition in the dispenser to be discharged for a predetermined period of dispensing time such as 5 seconds, 10 seconds, 15 seconds, 20 seconds, 30 seconds, 45 seconds, 1 minute, 1.5 minutes, 2 minutes, etc., each time the valve is actuated.

Alternatively, a conventional metered aerosol dispenser can be used for automatically and periodically dispensing this biologically-active composition into the atmosphere of the bathroom being treated. Metered aerosol dispensers of this type are well-known and commercially available from a wide variety of different sources including Hospeco of Cleveland, Ohio, Rubbermaid of Atlanta, Ga., LD Products of Long Beach, Calif., and BettyMills Company, Inc. of San Mateo, Calif. They are also described in many different patents including U.S. Pat. Nos. 2,980,302, 3,394,851, 5,921,447 and 7,387,121.

Normally, this assembly will be mounted so that it sprays the inventive biologically-active composition into the atmosphere of the bathroom to be treated in a manner so that the acetone and/or other carrier liquid in the composition, as well as the propellant in the dispenser, evaporate before reaching the floor or other hard surfaces being treated.

With this design, and because acetone or analog is used as the carrier liquid, contamination of the floor or other hard surface being treated with the carrier liquid is avoided, thereby avoiding the problems that can occur when water or ethanol, for example, are used as carrier liquids. In addition, odor problems are eliminated substantially completely, because a fresh supply of beneficial bacterial endospores is automatically and repeatedly applied to the hard surfaces which are, or may become, contaminated by urine and other wastes.

As indicated above, the persistent unpleasant odor that is often found in residential and commercial bathrooms is normally caused by naturally-occurring bacteria in the air digesting the urine and other waste products that may be present on various bathroom hard surfaces. In accordance with this invention, endospores of beneficial bacteria capable of digesting these waste products without creating obnoxious odors are applied to these hard surfaces in amounts which greatly exceed the amounts of naturally-occurring bacteria in the air. As a result, when these biologically-active materials, i.e., both the naturally-occurring bacteria in the air as well as the beneficial bacterial endospores of this invention, become biologically activated through contact with water, the vast majority of waste products on the hard surfaces carrying these wastes are consumed by the beneficial bacteria of this invention instead of the naturally-occurring bacteria in the air. The result is that little or no unpleasant odor is generated, since most if not essentially all of these waste products are converted into decomposition products which, themselves, exhibit little or no obnoxious odor.

Although only a few embodiments of this invention have been described above, it should be appreciated that many modifications can be made without departing from the spirit and scope of this invention. All such modifications are intended to be included within the scope of this invention, which is to be limited only by the following claims.

1. An aerosol spray dispenser comprising a container, an aerosol valve, and a pressurized aerosol composition inside the container comprising a biologically-active composition comprising a mixture of endospores of one or more beneficial bacteria capable of digesting dried urine without producing noxious odors and a carrier liquid for the endospores, wherein the carrier liquid comprises acetone.

2. The aerosol spray dispenser of claim 1, wherein the combined concentrations of water and ethanol in the composition is less than 10 wt. %, based on the weight of the composition as a whole, but excluding any propellant that might also be present.

3. The aerosol spray dispenser of claim 2, wherein the composition contains no more than trace amounts of water and ethanol.

4. The aerosol spray dispenser of claim 1, wherein the beneficial bacteria are spore-forming bacteria.

5. The aerosol spray dispenser of claim 1, wherein the beneficial bacteria are members of the phylum Firmicutes.

6. An aerosol system for eliminating the persistent unpleasant odor found in residential and commercial bathrooms, the aerosol system comprising the aerosol spray dispenser of claim 1 and an automatic dispensing assembly which is structured to automatically actuate the aerosol valve of the dispenser in accordance with a predetermined time pattern and, in addition, to mount this dispenser to spray the biologically-active composition contained therein into the atmosphere of the bathroom being treated in a manner so that the acetone in the composition, as well as the propellant in the dispenser, evaporate before reaching the floor or other hard surfaces of the bathroom.

7. The aerosol system of claim 6, wherein the aerosol system is a metered aerosol dispenser.

8. A process for eliminating the persistent unpleasant odors often found in commercial and residential bathrooms, the process comprising automatically and periodically discharging into the atmosphere of the bathroom being treated a biologically-active composition comprising a mixture of endospores of one or more beneficial bacteria capable of digesting dried urine without producing noxious odors and a carrier liquid for the endospores which comprises acetone in such a way that the endospores in this composition deposit on hard surfaces in the bathroom carrying dried urine while the acetone and propellant in this composition evaporate before reaching these hard surfaces.

9. The process of claim 8, wherein the biologically-active composition is automatically and periodically discharged into the atmosphere of the bathroom being treated by means of a metered aerosol dispenser.
